(19) United States
(12) Patent Application Publication
(10) Pub. No.: US 2009/0298743 A1
(43) Pub. Date: Dec. 3, 2009

(54) HYDROPHOBIC COMPOSITIONS CONTAINING RECONSTITUTION ENHANCER

(75) Inventors: Marc Besman, Seattle, WA (US); John Carpenter, Littleton, CO (US); Mark Manning, Loveland, CO (US); Lotte McNamara, Seattle, WA (US); Rajiv Nayar, Hercules, CA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/539,637

(22) Filed: Aug. 12, 2009

Related U.S. Application Data
(63) Continuation of application No. 10/925,797, filed on Aug. 25, 2004, now abandoned.
(60) Provisional application No. 60/500,908, filed on Sep. 5, 2003.

(51) Int. Cl. A61K 38/02; A61K 31/337; A61K 31/4375; A61P 43/00

(52) U.S. Cl. 514/2; 514/449; 514/283

(57) ABSTRACT

Disclosed are compositions comprising a hydrophobic active agent, a polymer and a reconstitution enhancing agent. Reconstitution of the lyophilized form of the compositions takes less time than in the absence of the enhancing agent.

### CROSS-REFERENCE TO RELATED APPLICATION

This application claims the benefit of U.S. application Ser. No. 10/925,797 filed Aug. 25, 2004, which claims the benefit of U.S. Provisional Application. No. 60/500,908, filed Sep. 5, 2003, which application is incorporated by reference herein in its entirety.

### BACKGROUND OF THE INVENTION

Hydrophobic drugs, including various anti-cancer drugs such as paclitaxel and its analog, docetaxel, are substantially insoluble in water or aqueous solution. Thus, paclitaxel has been formulated in a concentrated solution of 6 mg paclitaxel per milliliter in a carrier or vehicle containing Cremophor EL (polyoxyethylated castor oil) and dehydrated alcohol (50% v/v), which is then further diluted prior to administration. (Godspiel, 1994). Cremophor EL has been shown to cause toxic effects such as vasodilation, dyspnea and hypotension. (Ewiss et al., 1990). Accordingly, efforts have been made to avoid use of Cremophor in formulating paclitaxel. Some efforts have focused on the nature of the carrier. For example, there are several reports of formulating paclitaxel in a carrier that contains a tocopherol (vitamin E) and/or a vitamin E derivative such as Vitamin E TPGS. See, e.g., U.S. Pat. No. 6,358,373. Other efforts have focused on the active material, per se, and have resulted in the production of paclitaxel analogs, prodrugs and derivatives that are soluble in water. See, e.g., U.S. Pat. Nos. 6,344,571 and 6,175,023. In some cases, paclitaxel has been derivatized by way of conjugation with a poly-amino acid. See, e.g., U.S. Pat. Nos. 5,977,163; 6,262,107; 6,441,025; and 6,515,01 7, to Li, et al.

### SUMMARY OF THE INVENTION

One aspect of the present invention relates to a lyophilized composition of matter, comprising: (i) a hydrophobic biologically active molecule or active agent; (ii) a polymeric carrier that renders the active agent soluble in water, and (iii) a reconstitution enhancer or enhancing agent. Another aspect of the present invention is directed to an article of manufacture that contains the lyophilized composition of matter.

A further aspect of the present invention is directed to a method for decreasing the amount of time for a lyophilized composition to become reconstituted in an aqueous solution, comprising: preparing a lyophilized composition comprising a hydrophobic biologically active molecule or active agent; a polymeric carrier that renders the active agent soluble in water, and a reconstitution enhancer or enhancing agent, and adding the aqueous solution to the lyophilized composition, wherein the lyophilized composition becomes reconstituted in the aqueous solution in less time than in the absence of the reconstitution enhancing agent.

Applicants have discovered that the presence of a reconstitution enhancing agent in a lyophilized composition containing a hydrophobic active agent and a polymeric carrier allows the composition to be reconstituted in less time than in the absence of the agent. Embodiments of the present invention may also provide one or more additional advantages, namely, reduced need for vigorous agitation during reconstitution, less foaming (which otherwise can be excessive, entrapping the active agent and prolonging reconstitution time), reduced shrinkage of the lyocake (i.e., the lyophilized material), greater stability in physical characteristics of the lyophilized composition such as crystallinity, a robust lyophilization cycle with a cycle length of less than about 96 hours, and in some embodiments, as little as about 65 hours (i.e., lyophilization can be achieved in less time, and prolonged target shelf life.

### DETAILED DESCRIPTION OF THE INVENTION

Lyophilization is the process of removing water from a product by sublimation and desorption. Lyophilization equipment generally consists of a drying chamber with variable temperature control, a condenser to collect water removed from the product, and a vacuum system to reduce the pressure in the drying chamber. The lyophilization process generally consists of three stages: freezing, primary drying, and secondary drying. The temperature and pressure can be varied during the different stages to meet the various chemical and physical properties of the desired end product. The purpose of the freezing stage is to freeze the free water in the product. The rate of cooling can influence the structure of the frozen matrix. The pressure in the drying chamber is reduced and the temperature is increased for the primary drying phase that causes the frozen water to sublime. Following the first drying phase there is no longer any frozen water in the product. The secondary drying phase is designed to remove water that may be bound to the product. The temperature is further increased for this stage and the pressure may be changed, increased or decreased. Following secondary drying, the product is in its final lyophilized form.

Hydrophobic biologically active molecules are typically administered to human beings or other animals for therapeutic or diagnostic purposes. By the term “hydrophobic”, it is meant a molecule or active agent that in its non-ionized form is more soluble in lipid or fat than in water. See, U.S. Pat. No. 6,004,927. Typically, such agents are insoluble or substantially insoluble in water and/or aqueous solutions. Some representative examples of hydrophobic biologically active molecules are therapeutic agents, contrast agents and drugs such as taxanes (e.g., paclitaxel and docetaxel), etoposide, teniposide, fludarabine, doxorubicin, daunomycin, emodin, 5-fluorouracil, FUDR, estradiol, camptothecin, retinoic acids, verapamil, epothilones and cyclosporin. Anticancer drugs, specifically those from the taxane, camptothecin, epothilone, etoposide, and teniposide families, are suitable for use in the present invention. In preferred embodiments of the present invention, the hydrophobic active agent is a taxane or a camptothecin, and more preferably, paclitaxel, docetaxel or 20-S-camptothecin.

The polymer of the present invention is a carrier that allows the hydrophobic active agent to be soluble in aqueous solution. It is not intended to provide any additional therapeutic or diagnostic function to the compositions of the present invention (e.g., it is therapeutically and diagnostically inert). The polymer can be physically or chemically associated with the hydrophobic active agent in several ways. For example, it can simply be in admixture with the active agent; it can encapsulate or entrap the active agent or it can be attached or chemically coupled with the biologically active molecule such as via a covalent bond. See, e.g., U.S. Pat. Nos. 6,096,331; 6,365,191; 5,648,506; and 5,362,831. Representative examples of polymers include proteins, polypeptides, peptides and non-peptide polymers that are homopolymers or copolymers containing 2 or more different monomers. Examples include albumin, poly-alkylene glycols, polyvinyl alcohol, polyacrylates, polyhydroxyethyl methacrylate, polyacrylic acid, polyethyloxazoline, polyacrylamides, polyisopropyl acrylamides, polyvinyl pyrrolidinone, polyactide/glycolide, linear polyethylene glycols, branched polyethylene glycols, star polyethylene glycols, branched copolymers of polyethylene glycols with other functional monomers, polysaccharides, and combinations thereof. (Desai et al., 2003; Desai et al., 1997). Preferably the polymer is a peptide or polypeptide and/or hydrophilic. Preferred polymers include, but are not limited to polyethylene glycol, poly(1-glutamic acid), poly(d-glutamic acid), poly(dl-glutamic acid), poly(1-aspartic acid), poly(d-aspartic acid), poly(dl-aspartic acid), polyethylene glycol, copolymers of the above listed polyamino acids with polyethylene glycol, polycaprolactone, polyglycolic acid and polylactic acid, as well as polyacrylic acid, poly(2-hydroxyethyl 1-glutamine), carboxymethyl dextran, hyaluronic acid, human serum albumin and alginic acid, with polyethylene glycol, polyaspartic acids and polyglutamic acids being particularly preferred. The polyglutamic acids or polyaspartic acids of the present invention preferably have a molecular weight of about 5,000 to about 100,000 with about 20,000 to about 80,000, or even about 30,000 to about 60,000 and being about 32,000 preferred. Specifically, the most preferred polymer of the present invention is poly(L)-glutamic acid. See, U.S. Pat. Nos. 5,977,163; 6,262,107; 6,441,025; and 6,515,017, all to Li, et al. The relative amounts of hydrophobic active agent and polymer present in the compositions of the present invention may be determined in accordance with standard procedures, taking into account such factors as the intended use of the composition, the nature of the active agent and polymer, and the manner in which they are associated.

A reconstitution enhancer is a material which, when added to or contained in a lyophilized composition, causes the lyophilized composition to dissolve in water and/or aqueous solution more quickly than said lyophilized composition would dissolve without the reconstitution enhancer. In the present invention, it is preferred that the reconstitution enhancer is lyophilized with the hydrophobic biologically active molecule and the polymer. Reconstitution enhancing agents suitable for use in the present invention include disaccharides such as sucrose and trehalose. Mannitol is further a suitable enhancing agent. These excipients have been disclosed as useful in one or more aspects of stabilizing biologically active proteins in lyophilized form. Specifically, sucrose and trehalose are known to reduce protein unfolding and aggregation, stabilize amorphous phase components of lyophilized compositions containing biologically active proteins (which include, among others, the protein, amorphous excipients and water), and along with various other excipients such as other sugars, polyols, certain amino acids, methylamines and salting out salts, stabilize proteins during freezing or freeze-thawing. See, Carpenter, et al., “Rational Design of Stable Lyophilized Protein Formulations: Theory and Practice,” Chapter 5 in “Rational Design of Stable Protein Formulations Theory and Practice, Vol. 13 in Pharmaceutical Biotechnology (Carpenter, et al., eds.), Kluwer Academic/Plenum Pub. (New York), 2002. Carpenter also discloses the inclusion of bulking agents e.g., mannitol, glycine and hydroxylethyl starch (HES), in such lyophilized compositions. Protein stabilizers are also disclosed in Arakawa, et al., Adv. Drug Del. Rev. 46:307-326 (2001) (disclosing protein stabilizers including sugars (sucrose, lactose and glucose), amino acids (glycine, alanine and proline), amines (betaine and trimethylamine N-oxide), polyols (mannitol and sorbitol) and certain salts (ammonium, sodium and magnesium sulfate)).

With respect to reconstitution, however, Applicants have shown that glycine and HES are not suitable as reconstitution enhancing agents in the present invention. Likewise, Zou, et al., Cancer Chemother. Pharmacol. 39(1/2):103-108 (1996), reports that the presence of Tween 20 in a lyophilized composition containing phospholipids and the anthracycline drug annamycin, was essential in shortening the reconstitution step from less than two hours to 1 minute and avoided the early formation of free drug crystals, and reducing the median particle size without destroying the liposome vesicles. Applicants have found, however, that Tween 80, a related surfactant, did not enhance reconstitution in some embodiments in which it was tested. It may be used, however, in connection with other reconstitution enhancing agents, e.g., as discussed below. Thus, it was not predictable that protein stabilizers would function as reconstitution enhancing agents in the context of the present invention. Nonetheless, persons skilled in the art may determine whether a given protein stabilizer e.g., a sugar, disaccharide, polyol, amino acid, amine, salt or bulking agent, will function as a reconstitution enhancing agent for purposes of the present invention simply by testing it e.g., in accordance with the protocols set forth in the examples section.

In some preferred embodiments of the present invention, the reconstitution enhancer comprises mannitol. The present invention is not limited to compositions containing one reconstitution enhancing agent, however. In other preferred embodiments, the compositions contain combinations of mannitol and trehalose. Amounts of the one or more reconstitution enhancing agents generally range from about 0.5% to about 10% by total weight of the lyophilized composition. In yet other embodiments, surfactants such as SDS, Tween 20 and Tween 80 may be added in relatively small amounts (e.g., 0.005 to 0.2%, and preferably from about 0.05 to about 0.1%) to reduce foam that is often caused by the agitation involved during reconstitution. In some embodiments, the addition of Tween 80 will further reduce reconstitution time.

The compositions of the present invention may contain other inert pharmaceutically acceptable ingredients, such as buffering agents (e.g., phosphate buffers), amino acids (e.g., glycine, arginine, histidine), salts (e.g., sodium chloride), polymers (e.g., polyethylene glycols) or other bulking or carrier agents. The compositions of the present invention can be contained in a variety of containers such as vials and syringes. Packages containing these containers may also contain an additional container containing a volume of water e.g., bacteriostatic water, for reconstitution of the lyophilized composition.

Persons skilled in the art will also appreciate that adjustment of other parameters associated with the lyophilization procedure may have a positive effect on the composition. For example, compositions containing mannitol as a reconstitution enhancing agent might show increased moisture levels due to formation of mannitol hydrate, thus potentially affecting storage stability. In these cases, moisture levels of the lyophilized composition may be reduced e.g., by lyophilizing the composition at a relatively high secondary drying temperature e.g., about 40° C.-50° C. The lyophilization cycle might be reduced by optimizing annealing parameters that influence primary drying duration. Annealing at higher temperatures results in Ostwald ripening, i.e., formation of larger ice crystals, which in turn allows for increased sublimation rates during primary drying. Additionally, reduction in reconstitution time might be achieved by varying the molarity (concentration) of a pharmaceutically inert ingredient contained in the composition, or a chemical property of the composition, such as pH. For instance, in embodiments shown in the examples (e.g., compositions containing an ester conjugate of α-poly-(L)-glutamic acid and paclitaxel, preferably covalently bonded at the 2′ hydroxyl site on paclitaxel), reconstitution may be further optimized by adjusting pH (e.g., in a range of 5.4-5.8. preferably 5.7), and if a buffer is used, having it present in an amount of from 150 to 220 mM, preferably 200 mM.

The invention will be further described by reference to the following detailed examples. These examples are provided for purposes of illustration only, and are not intended to limit the scope of the invention described herein.

### EXAMPLES

### Materials

CT-2103 is the designation for the active pharmaceutical ingredient (API) used in the following examples. CT-2103 is the ester conjugate of α-poly-(L)-glutamic acid (PG), and paclitaxel, primarily bound at 2′ hydroxyl site on paclitaxel. The base PG polymer is about 17,000 Daltons (apparent average molecular weight by gel permeation chromatography and multi-angle laser light scattering). Paclitaxel is present in the bound form at about 37% (32% to 42% loading, wt/wt) in the conjugate, equivalent to about one paclitaxel ester linkage per 11 monomer units of the polymer. The final product (FP) formulation consists of 9 mg/mL conjugated paclitaxel (≈25 mg/mL CT-2103) with 260 mM phosphate buffer at pH 6.0, and 0.5% w/w Poloxamer 188 (F-68) (e.g., triblock copolymer poly(ethelene) oxide-poly(propylene) oxide-poly(ethylene) oxide). The FP is reconstituted to 9 mg paclitaxel/mL (25 mg CT-2103 API/mL) with sterile water for injection, USP.

Sucrose (cat S124-1, lot #28409A), trehalose (cat T104-1, lot #27881A), mannitol (cat M109-2, lot #27214A) were obtained from Pfanstiehl. Poloxamer 188, NF (F-68) was obtained from BASF (lot #WPDX-577B). Sodium phosphate monobasic, monohydrate, USP (cat. SO130) and sodium phosphate dibasic, heptahydrate, USP (cat. SO140) were purchased from Spectrum chemicals. Water was purified using the MilliQ system from Millipore.

Schott glass vials (obtained from West Co.) and West gray stoppers 4432/50 were used. Two vial sizes were utilized in the studies, 5 mL and 20 mL size with 20 mm opening. Studies using the 2 mL fill volume and 10 mL fill volume were performed in the 5 mL and 20 mL vial sizes, respectively. Vials were rinsed at least three times with water and dried before use. Stoppers were used without any further processing.

### Experimental Design Methodology and Analysis

ECHIP Design-of-Experiments (DOE) software was utilized to generate the experimental design, analyze, and interpret the data. A response surface quadratic design was selected for the experimental trials. Such designs offer optimal number of experimental trials and analyzed results provide rationale for the various excipients and their concentrations, and good visualization of interactions that may exist among the experimental variables being evaluated. The data were also analyzed using principle latent structure (PLS) analysis software to validate the conclusions drawn from ECHIP by an orthogonal method. All designs met or exceeded the experimental G-efficiency of at least 50%, which was a measure of quality in the experimental design.

### Secondary Structure by Far UV Circular Dichroism

The purpose for performing the far UV circular dichroism was to obtain secondary structural information from the reconstituted lots of CT2103 FP. Far UV circular dichroism (FUV-CD) was utilized to characterize the structure of different CT-2103 lots. Far UV circular dichroism (CD) spectra were collected on an Aviv model 62 DS spectropolarimeter (Lakewood, N.J.) for the samples. Each sample was loaded into a 0.1 cm path-length quartz cell and placed in a thermostated cell holder. Data were collected at 0.5 um intervals utilizing a 1.5 nm bandwidth, with an averaging time of 5 seconds at each point. The appropriate buffer blank was collected and subtracted from each spectrum.

### Second Derivative Ultraviolet Spectroscopy

Second derivative spectroscopy was used to obtain tertiary structural information from the reconstituted CT-2103 FP. Second derivative ultraviolet spectroscopy was performed to compare any subtle structural features that might occur in different lots of CT-2103 due to the local microenvironments of the absorbing chromophores. UV data were collected in a 1 cm path length quartz cell on a Hewlett Packard 8452A diode array spectrophotometer with a 25 second integration time. The data is imported into Grains 386 software. The data is truncated to 2.15-350 nm, the second derivative is processed using the Savitszky-Golay transformation. The curve is smoothed using a 3 data point window and over 7 points and the data points are interpolated to a spline function of 32X.

### Fourier Transform Infrared Spectroscopy (FTIR)

FTIR was utilized to characterize secondary structural information in the solid states of CT-2103 FP lots. Infrared spectra were obtained with a Bomem Prota FTIR spectrometer (Quebec City, Quebec, Canada) equipped with a DTGS detector. The instrument was continually purged with dry air. For each sample, a total of 128 interferograms were collected and averaged using a resolution of 4 cm⁻¹. The dried protein spectra were collected in single-beam transmission mode and ratioed against dry air to create the absorbance spectra. A second derivative spectrum of water vapor was subtracted to remove water vapor interference, when necessary. Second derivative spectra were created using the Savitzky-Golay function of second degree polynomial with a 7-point window, using Bomem Grams/32 software; (Galactic Industries). FTIR was performed on solid samples representing 6 different lots of CT-2 103 FP.

### High Temperature Differential Scanning Calorimetry

High temperature DSC was performed on a Perkin-Elmer Diamond DSC instrument to monitor any crystallization and glass-transition events in the solid state. The samples were scanned typically at 25° C./min to 100° C., cooled to −20° C. and rescanned to 150° C. In order to improve the measurement of the glass transition temperature in the FP, StepScan DSC technique was employed which reduces interferences from other thermal events such as crystallization, enthalpic relaxation, and loss of moisture and provides a clear glass transition temperature, which is a reversible event, rather than a kinetic or irreversible event on the time scale of this analysis.

### Low Temperature Differential Scanning Calorimetry (DSC)

Differential scanning calorimetry of frozen solutions was carried out with a Seiko DSC 6100 using liquid nitrogen cooling. The DSC instrument was calibrated with an indium standard. Samples of reconstituted solution (20 μL or 50 μL of a 9 mg paclitaxel/mL solution) were placed in aluminum sample pans and hermetically sealed for thermal analysis. The reference used with all samples was an empty aluminum pan. Nitrogen was used as the purge gas at a rate of 50 mL/min. Samples were frozen at a controlled rate of 2° C./min to approximately −70° C. and warmed to room temperature at 2° C./min. Thermograms were recorded during freezing and warming of the sample.

Data were captured at 0.5 sec intervals and analyzed using Seiko software. Thermal transition temperatures, such as the super-cooling temperature (T_{sc}), glass transition temperature of the frozen solution (T_{g}′), recrystallization temperature (T_{cr}), and the eutectic melt temperature (Tₑᵤ) were determined from the heat flow data.

### Lyophilization

Lyophilization was performed using a Genesis Pilot-scale Virtis 1 2XL lyophilizer equipped with three stoppering shelves and an external condenser. Parameters such as ramp rate, shelf-temperature, time, and vacuum were programmed into the cycle run and the product temperatures were recorded using four available thermocouples by the Wizard control system software provided by the Virtis Company. The data was subsequently processed and graphed using IGOR scientific graphing software.

### Reconstitution Method

Reconstitution of the FP was assessed by adding MilliQ purified water to the side of the vial that had been-stoppered under vacuum. After standing for 30 seconds, percent cake hydration was estimated visually and the vial was swirled gently until all visible solids had dissolved. The final reconstitution time was recorded as the total time required for the cake to dissolve into a clear solution (including the 30 seconds to estimate cake hydration). In some cases, the product hydrated fully and dissolved into a clear solution in less than 30 seconds. Since the amount of foam could vary between experiments, vials were grouped into five groups with varying degree of foam and assigned the grading scale of 1 to 5, where 1 represented little/no foam and 5 represented to some foam.

### X-ray Diffraction (XRD)

XRD was performed on lyophilized CT-2 103 FP to obtain information on crystallinity of the product and characterization of the different polymorphs of the formulation components. The lyophilized product was filled in an aluminum holder, by the side-drift method, and exposed to CuKα radiation (45 kV×40 mA) in a wide-angle X-ray powder diffractometer (Model D5005, Siemens). The instrument was operated in the step-scan mode, in increments of 0.05° 2θ. The angular range was 5° to 40° 2θ and counts were accumulated for 1 sec at each step. The data collection program used was JADE 5.0.

### Scanning Electron Microscopy (SEM)

SEM was performed to visualize the cake structures of different CT-2103 formulations and find possible correlations with the reconstitution properties in various lyophilized formulations. The CT-2103 FP was processed for SEM by rapidly cutting the lyophilized cakes into pieces using a freshly made clean bamboo stick (Electron Microscopy Sciences, Pa.). The pieces were attached to a 12 mm OD aluminum SEM specimen-mounting stub by spreading a thin layer of the sample over a double-sided carbon conductive tab that was attached to the stub. This was performed rapidly (1-2 min) at room temperature to avoid any artifacts from sample handling. The sample stub was quickly transferred to a sputter coater and placed under vacuum. The samples were coated using a sputter coater (Biorad E5000M) with approximately 40 nm gold/palladium. Examination of samples was performed with a Hitachi A60 field emission SEM operating at 10 kV.

### Residual Moisture Analysis

Residual moisture was determined by the Karl Fisher coulormetry method following extraction of water from the lyophilizate with anhydrous methanol. This technique minimized any artifacts as a result of direct sample handling and exposure to atmospheric humidity. Anhydrous methanol was added to the vial containing lyophilized CT-2 103 FP and the cake was suspended in the methanol by vortexing. Undissolved excipients were allowed to settle for about 1 hour and the methanol containing the extracted water from the cake was analyzed for moisture content. Anhydrous methanol solution was used to subtract the background water content in the methanol.

### Bioanalyzer Protein Chip

Protein Chip sizing technique, which provides a high throughput analytical method for SDS-PAGE analysis, was employed to analyze reconstituted CT-2 103 FP samples. The principle is based on micro-fluidic capillary electrophoresis. Samples were analyzed using the Protein 200 LabChip® from Agilent.

### Formulation Studies

A number of pharmaceutically acceptable generally recognized as safe (GRAS) excipients including mannitol and Tween-80 containing formulations. Thirteen formulation matrices were evaluated in addition to the control F-68 formulation (Table 1). Formulations CT-1 to CT-14 were prepared by dissolving the CT-2103 active pharmaceutical ingredient (API) and excipients in 200 mM sodium phosphate, pH 6.5 at 50° C.-55° C. Solutions were filtered through a 0.22 μm GV Millipore filter and 2 mL was filled into 5 mL Schott Type I glass vials, which were lyophilized using a conservative cycle with an annealing step at −10° C. and primary drying at −25° C. for 30 hours followed by secondary drying at +20° C. for not less than 10 hours. Annealing was incorporated into the freezing phase to promote any crystallization of excipients that were amenable to crystallization from the amorphous phase.

TABLE I
Formulation sample composition. pH values denote pH
of the buffer prior to API addition.

| | | | | | | | Sodium | |
|---|---|---|---|---|---|---|---|---|
| Formula# | Sucrose | Trehalose | Mannitol | Glycine | F-68 | TW-80 | Phosphate | pH |
| CT-1 | 5.0% | | | | | | 200 mM | 6.5 |
| CT-2 | 5.0% | | | | 0.5% | | 200 mM | 6.5 |
| CT-3 | 5.0% | | | | | 0.1% | 200 mM | 6.5 |
| CT-4 | 1.0% | | 4.0% | | | 0.1% | 200 mM | 6.5 |
| CT-5 | 1.0% | | | 2.5% | | 0.1% | 200 mM | 6.5 |
| CT-7 | | 5.0% | | | | 0.1% | 200 mM | 6.5 |
| CT-8 | | 1.0% | 4.0% | | | 0.1% | 200 mM | 6.5 |
| CT-9 | | 1.0% | | 2.5% | | 0.1% | 200 mM | 6.5 |
| CT-11 | | | 4.0% | | | 0.1% | 200 mM | 6.5 |
| CT-12 | | | | 2.5% | | 0.1% | 200 mM | 6.5 |
| CT-14 | | | | | 0.5% | 0.0% | 200 mM | 6.5 |

Reconstitution characteristics were evaluated by adding 2 mL of water, noting the cake hydration after 30 seconds or less followed by gently swirling the vial contents until all the solids were fully dissolved. Results are shown in Table II (wherein S=sucr=sucrose, TW-tween-80. G=glycine, T=trehalose, M=mannitol, and the number preceding the letter designates percent w/v).

TABLE II
Reconstitution characteristics of CT-2
103 in different formulation matrices.

| Formula | | | Recon | |
|---|---|---|---|---|
| # | Formulation | Hydration characteristics | time (sec) | |
| CT-1 | 5% Sucr | 100% hydrated in 20 sec | 60 | sec |
| CT-2 | 5S/F68 | 100% hydrated in 20 sec | 50 | sec |
| CT-3 | 5S/TW8O | 100% hydrated in 20 sec | 60 | sec |
| CT-4 | 1S/4M/TW | 100% hydrated in 20 sec | 30 | sec |
| CT-5 | 1S/2.5G/TW | 0% hydrated | 100 | sec |
| CT-7 | 5T/TW8O | 30% hydrated | 75 | sec |
| CT-8 | 1T/4M/TW | 100% in 6 sec | 10 | sec |
| CT-9 | 1T/2.5G/TW | 0% hydrated | 105 | sec |
| CT-11 | 4M/TW | 100% in 10 sec | 30 | sec |
| CT-12 | 2.5G/TW | 0% hydrated | 95 | sec |
| CT-14 | Control 0.5% F68 | 90% hydrated | 75 | sec |

Formulations CT4, -8 and -11 showed relatively fast reconstitution times compared to control and other formulations. The results showed that mannitol greatly enhanced the reconstitution properties of CT-2103. Sucrose and trehalose also enhanced reconstitution times, whereas the role of Tween-80 appeared to be formulation dependent, and thus not useful as a sole reconstitution enhancing agent. On the other hand, glycine clearly did not offer any advantage and appeared to have a negative influence on the reconstitution of CT-2103.

In the next series of experiments, these three formulations (i.e., CT-4, -8, -11) were further studied. with and without the addition of Tween-80. The effect of hydroxyethyl starch (HES) was also evaluated. The formulations were prepared at the final dosage form of 10 mL/vial and evaluated for reconstitution time and foam characteristics. The results are summarized in Table III (wherein S=sucrose, T=trehalose, M=mannitol, HE=hydroxyethyl starch, TW=Tween-80, control=0.5% F-68, and the number designates percent level of the formulation).

TABLE III
Role of Tween-80 and HES on reconstitution of CT-2 103.

| | | | Recon- | | |
|---|---|---|---|---|---|
| Formula | | | stitution | | |
| 4 | Formulation | Hydration characteristics | time (sec) | | Foam |
| CT-4 | 1S/4M | 95% hydrated in 20 sec | 60 | sec | 5 |
| CT-4Tw | 1S/4M/TW | 100% hydrated in 20 sec | 50 | sec | 4 |
| CT-8 | 1T/4M | 100% in 8 sec | 8 | sec | 5 |
| CT-8Tw | 1T/4M/TW | 95% in 20 sec | 40 | sec | 4 |
| CT-6Tw | 1S/2.5HES/TW | 100% hydrated but | 150 | sec | 3 |
| | | particulate | | | |
| CT-11 | 4M | 100% in 20 sec | 47 | sec | 4 |
| CT-11Tw | 4M/TW | 100% in 20 sec | 140 | sec | 1 |
| CT-13Tw | 2.5HES/TW | 100% hydrated but | 120 | sec | 2 |
| | | particulate | | | |
| CT-14 | Control 0.5% | 10% hydrated | 372 | sec | 3 |
| | F68/4M | | | | |
| CT-14B | 0.5% F68/4M | Hydrated but settled | 285 | sec | |
| | | on bottom | | | |

The results of this study showed that HES did not enhance reconstitution of CT-2103 relative to trehalose or sucrose. Tween-80 appeared to increase the reconstitution time, when used in the mannitol-sugar formulations. The trehalose/mannitol formulation reconstituted significantly faster than the sucrose/mannitol formulation (8 seconds compared to 60 seconds). The control (CT-14) exhibited a long reconstitution time of around six minutes. Addition of mannitol to this formulation only slightly increased the reconstitution properties of CT-2103. The foam characteristics of the formulations were graded on a scale of 1 to 5, where 1 was no foam and 5 was some foam.

These formulation results showed that disaccharides (sucrose or trehalose), mannitol, and Tween-80 improved reconstitution characteristics of CT-2103.

In order to evaluate the role of these excipients, their relative concentrations, and any interactions in the formulation matrix, a multivariable statistical experimental design was performed using the following variables, as shown in Table IV.

TABLE IV
Design of experiments evaluating various potential
excipients on CT-2103 reconstitution.

| Formu- | Trehalose | Mannitol | TW-80 | F-68 | Sodium |
|---|---|---|---|---|---|
| lation | (%) | (%) | (%) | (%) | Phosphate |
| CT-22 | 5.0 | 2.0 | | | 200 mM |
| CT-30 | 5.0 | | 0.05 | | 200 mM |
| CT-34 | 5.0 | 2.0 | 0.10 | | 200 mM |
| CT-27 | | 2.0 | 0.05 | | 200 mM |
| CT-24 | | | 0.10 | | 200 mM |
| CT-23 | | 4.0 | | | 200 mM |
| CT-25 | 5.0 | 4.0 | 0.10 | | 200 mM |
| CT-29 | | | | 0.50 | 200 mM |
| CT-32 | 2.5 | 4.0 | | | 200 mM |
| CT-35 | | 2.0 | | | 200 mM |
| CT-33 | 2.5 | | | | 200 mM |
| CT-21 | | 4.0 | 0.10 | | 200 mM |
| CT-25B | 5.0 | 4.0 | 0.10 | | 200 mM |
| CT-21B | | 4.0 | 0.10 | | 200 mM |
| CT-22B | 5.0 | 2.0 | | | 200 mM |
| CT-31 | 5.0 | 4.0 | 0.05 | | 200 mM |
| CT-28 | 2.5 | 2.0 | 0.10 | | 200 mM |
| CT-24B | | | 0.10 | | 200 mM |
| CT-26 | 2.5 | 4.0 | 0.05 | | 200 mM |
| CT-23B | | 4.0 | | | 200 mM |

| | |
|---|---|
| NEAT | 200 mM |

All formulations were produced with 200 mM sodium phosphate buffer, at pH 6.5 (prior to mixing with-API). (NEAT=CT-2103 dissolved in 200 mM sodium phosphate buffer.)

This series of experimental trials were performed at the 2 mL/vial dosage form. Two vials were reconstituted and evaluated for percent hydration at 20-30 seconds, total reconstitution time (seconds), and foam (scale 1-5). Results for this study are shown in Table V.

TABLE V
Results from the DOE shown in Table IV.

| | Vial 1 | | | Vial 2 | | |
|---|---|---|---|---|---|---|
| | | reconstitution | | | reconstitution | |
| | hydration | time | foam | hydration | time | foam |
| Formulation | % | (secs) | (1 to 5) | $ | (secs) | (1 to 5) |
| CT-22 | 60 | 170 | 2 | 75 | 100 | 3 |
| CT-30 | 10 | 170 | 2 | 10 | 220 | 2 |
| CT-34 | 40 | 120 | 5 | 95 | 65 | 2 |
| CT-27 | 85 | 130 | 1 | 90 | 75 | 1 |
| CT-24 | 5 | 126 | 1 | 70 | 120 | 4 |
| CT-23 | 95 | 60 | 3 | 95 | 70 | 2 |
| CT-25 | 90 | 62 | 4 | 95 | 80 | 5 |
| CT-29 | 5 | 170 | 5 | 10 | 130 | 4 |
| CT-32 | 80 | 100 | 4 | 95 | 70 | 2 |
| CT-35 | 75 | 120 | 1 | 85 | 120 | 3 |
| CT-33 | 0 | 220 | 5 | 5 | 240 | 5 |
| CT-21 | 85 | 75 | 5 | 95 | 75 | 1 |
| CT-25B | 80 | 60 | 2 | 95 | 80 | 1 |
| CT-21B | 5 | 150 | 2 | 85 | 80 | 2 |
| CT-22B | 60 | 110 | 5 | 75 | 110 | 2 |
| CT-31 | 95 | 62 | 4 | 95 | 60 | 3 |
| CT-28 | 5 | 170 | 2 | 5 | 170 | 2 |
| CT-24B | 95 | 80 | 1 | 90 | 90 | 1 |
| CT-26 | 95 | 70 | 3 | 95 | 80 | 1 |
| CT-23B | 95 | 75 | 4 | 90 | 85 | 5 |
| NEAT | 0 | 165 | 5 | 10 | 170 | 4 |

The results were analyzed using ECHIP. The results (data not shown) indicate that mannitol was the most important variable for reconstitution time and was negatively correlated to the reconstitution time, suggesting that higher levels of mannitol give faster reconstitution times for CT-2103. Also, the results suggested that there was an interaction between mannitol and Tween-80 that was positively correlated to reconstitution time, suggesting that addition of Tween-80 to the mannitol formulation may increase the reconstitution time of CT-2103. The results further showed that varying the ratio of trehalose to mannitol may affect reconstitution time.

The responses were optimized for minimum reconstitution time, minimum foam, and maximum hydration and an optimum formulation was predicted from the experimental design. The design (not shown) predicted that an even more preferred formulation of CT-2103 could be comprised of 4% mannitol, 5% trehalose and 0.05% Tween-80.

Based on these results, formulations of CT-2103 were scaled up to the final dosage form of 10 mL/vial and evaluated for percent hydration, reconstitution time, and foam. These results suggested that ECHIP closely predicted the lead formulations from the experimental design study and that the two more preferred formulations contained 4% mannitol and 4% mannitol/5% trehalose/0.1% Tween-80.

These formulations, together with the control, were further characterized by XRD. All mannitol-based formulations appeared to contain crystalline delta polymorph of mannitol. A small quantity of mannitol hydrate was observed in the 4% mannitol formulation (CT-31) as evidenced by the peaks at 9.6°, 17.9°, 25.7°, and 27.0° 2θ. This formulation exhibited the greatest degree of crystallinity of all the formulations. The pure trehalose and the control F-68 formulation were predominantly amorphous. However, the presence of unassigned peaks in the F-68 formulation suggests some degree of crystallinity. The results are summarized in Table VI.

TABLE VI
Summary of XRD results.

| Sample | Sample | |
|---|---|---|
| code | composition | Phases Identified |
| CT-31 | 4% Mannitol | Anhydrous δ polymorph of mannitol. |
| | | Mannitol hydrate* |
| CT-32 | 4% Mannitol, | Anhydrous δ polymorph of mannitol. |
| | 5% Trehalose | Very small amount of mannitol |
| | | hydrate* |
| CT321T1 | 4% Mannitol, | Anhydrous δ polymorph of mannitol. |
| | 5% Trehalose, | |
| | 0.05% Tween-80 | |
| CT-33 | 5% Trehalose | Amorphous lyophile. |
| CT-33 T1 | 5% Trehalose, | Amorphous lyophile. |
| | 0.05% Tween-80 | |
| CT-34 | F68 (0.5 mg/mL) | X-ray diffraction peaks could not |
| | | be assigned. |

*The intensities of the peaks attributable to the mannitol hydrate in CT-31 and CT-32 XRD patterns suggested that it was a minor component of the formulation components.

All publications cited in the specification (e.g., the list of citations below) are indicative of the level of skill of those skilled in the art to which this invention pertains. All these publications are herein incorporated by reference to the same extent as if each individual publication were specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A lyophilized composition comprising a hydrophobic biologically active agent; a polymer that renders said hydrophobic active agent soluble in an aqueous solution, and a reconstitution enhancing agent in an amount from about 0.5% to about 10% by total weight of the lyophilized composition, wherein time of reconstitution of said composition in an aqueous solution is less than that for said composition absent said enhancing agent.

2. The composition of claim 1, wherein said hydrophobic active agent comprises a taxane.

3. The composition of claim 1, wherein said hydrophobic active agent comprises paclitaxel.

4. The composition of claim 1, wherein said hydrophobic active agent comprises docetaxel.

5. The composition of claim 1, wherein said hydrophobic active agent comprises a camptothecin.

6. A lyophilized composition comprising a hydrophobic taxane; a polymer that renders said taxane soluble in an aqueous solution, and a reconstitution enhancing agent in an amount from about 0.5% to about 10% by total weight of the lyophilized composition, wherein time of reconstitution of said composition in an aqueous solution is less than that for said composition absent said enhancing agent.

7. The composition of claim 6, wherein said taxane is paclitaxel.

8. The composition of claim 6, wherein said taxane is paclitaxel, and said polymer comprises a peptide whose majority of amino acid residues are glutamic acid residues, wherein said paclitaxel and said polymer are in the form of a conjugate.
